(19) United States
(12) Patent Application Publication
(10) Pub. No.: US 2024/0298923 A1
(43) Pub. Date: Sep. 12, 2024

(54) ANATOMICAL LUMEN SIZING DEVICE

(71) Applicant: GYRUS ACMI, INC. D/B/A OLYMPUS SURGICAL TECHNOLOGIES AMERICA, Westborough, MA (US)

(72) Inventor: Joey Magno, Dudley, MA (US)

(21) Appl. No.: 18/590,547

(22) Filed: Feb. 28, 2024

Related U.S. Application Data
(60) Provisional application No. 63/489,218, filed on Mar. 9, 2023.

(51) Int. Cl. A61B 5/107; A61B 5/00; A61B 90/00

(52) U.S. Cl. CPC A61B 5/1076; A61B 5/6859; A61B 2090/0811

(57) ABSTRACT

A sizing device configured to measure a dimension of a lumen in a patient can include an elongated member, an extendable assembly, and an actuation mechanism. The elongated member can be insertable into the patient and define a lumen extending from a proximal end to a distal end. The extendable assembly can be insertable into the lumen. The extendable assembly can include elements that can have a shape-set memory bias. The shape-set memory bias can be configured to allow the elements to extend radially outward from a central axis of the elongated member. The actuation mechanism can be operably coupled to the extendable assembly and can be configured to move the elements between a retracted position and a plurality of deployed positions.

### CLAIM OF PRIORITY

This patent application claims the benefit of priority, under 35 U.S.C. Section 119 (e), to Joey Magno U.S. patent application Ser. No. 64/489,218, entitled “ANATOMICAL LUMEN SIZING DEVICE,” filed on Mar. 9, 2023 (Attorney Docket No. 5409.832PRV), which is hereby incorporated by reference herein in its entirety.

### TECHNICAL FIELD

Embodiments described herein generally relate to a sizing device and, more specifically, to a sizing device for finding a dimension of a lumen of a patient.

### BACKGROUND

Sizing devices can be used to measure a dimension of a lumen, airway, artery, or any other passageway within a patient. The sizing device can help a medical professional know the size of the lumen before inserting another medical device (e.g., a balloon, stent, valve, or the like) within the patient. Thus, the sizing device can be important to help the medical professional determine an appropriate size of medical device that should be inserted into the lumen of the patient.

### SUMMARY

In an example, a sizing device can be configured to measure a dimension of an airway in a patient. The sizing device can include an elongated member, an extendable assembly, and an actuation mechanism. The elongated member can be insertable into a lung of the patient and can define a lumen extending therethrough from a proximal end to a distal end. The extendable assembly can be insertable into the lumen. The extendable assembly can include wires having a shape-set memory bias. The shape-set memory bias can be configured to allow the wires to extend radially outward from a central axis of the elongated member. The actuation mechanism can be operably coupled to the extendable assembly and can be configured to move the wires between a retracted position and a plurality of successive deployed positions. In the retracted position, the wires can be disposed within the lumen. In the plurality of deployed positions, the wires can extend distally beyond the distal end of the elongated member. The shape-set memory bias can cause the wires to extend radially outward to define a plurality of successive testing dimensions.

In another example, a sizing device can be configured to measure a dimension of a lumen in a patient, the sizing device can include an elongated member, an extendable assembly, and an actuation mechanism. The elongated member can be insertable into the patient and can define a lumen extending therethrough from a proximal end to a distal end. The extendable assembly can be insertable into the lumen and can include elements with a shape-set memory bias. The shape-set memory bias can be configured to allow the elements to extend radially outward from a central axis of the elongated member. The actuation mechanism can be operably coupled to the extendable assembly and can be configured to move the elements between a retracted position and a plurality of deployed positions. In the retracted position, the elements can be disposed within the lumen. In the plurality of deployed positions, the elements can extend distally beyond the distal end of the elongated member, and the shape-set memory bias can cause the elements to extend radially outward to define a plurality of testing dimensions.

In yet another example, a method for reprocessing a sizing device of any of the other examples. The method can include obtaining the sizing device. The method can include sterilizing the sizing device. The method can also include storing the sizing device in a sterile container.

### BRIEF DESCRIPTION OF THE DRAWINGS

Various examples are illustrated in the figures of the accompanying drawings. Such examples are demonstrative and not intended to be exhaustive or exclusive embodiments of the present subject matter.

FIG. 1 illustrates a portion of a sizing device, according to an example of the present disclosure.

FIG. 2 illustrates a partial cut-away view of a distal portion of an elongated member of a sizing device, according to an example of the present disclosure.

FIG. 3 illustrates a front view of a distal tip of an elongated member of a sizing device, according to an example of the present disclosure.

FIG. 4A illustrates a schematic diagram of a sizing device within a lung of a patient, according to an example of the present disclosure.

FIG. 4B illustrates an enlarged view of a portion of the sizing device of FIG. 4A indicated by label 4B-4B, according to an example of the present disclosure.

FIG. 5A illustrates a side view of a partial cut-away view of a distal end of an elongated member of a sizing device in a first position of a plurality of deployed positions, according to an example of the present disclosure.

FIG. 5B illustrates a front view of a distal end of an elongated member of a sizing device in the first position of a plurality of deployed positions, according to an example of the present disclosure.

FIG. 6A illustrates a side view of a partial cut-away view of a distal end of an elongated member of a sizing device in a second position of a plurality of deployed positions, according to an example of the present disclosure.

FIG. 6B illustrates a front view of a distal end of an elongated member of a sizing device in the second position of a plurality of deployed positions, according to an example of the present disclosure.

FIG. 7 illustrates an exploded view of a portion of a sizing device, according to an example of the present disclosure.

FIG. 8 illustrates a partial cut-away view of another sizing device, according to an example of the present disclosure.

FIG. 9 illustrates a flow diagram of a method for reprocessing a sizing device, according to an example of the present disclosure.

### DETAILED DESCRIPTION

Sizing devices can be used to determine a size of a lumen, airway, artery, or any other passageway within a patient. The sizing device can help a medical professional confirm the size of the lumen before inserting another medical device (e.g., a balloon, stent, valve, or the like) within the patient. Thus, the sizing device can be important to help the medical professional determine an appropriate size of medical device that should be inserted into the lumen of the patient. For example, prior to placing endobronchial valves that restrict airflow to diseased or otherwise damaged lung volumes (e.g., emphysematous lung regions or lobes), a medical professional typically must determine which lung volumes to restrict airflow to and the sizes for any airways leading to such volumes, so that an appropriately sized endobronchial valve may be placed.

To accomplish the aforementioned planning for an airway valve placement procedure, a conventional airway valve planning tool may require performing multiple steps to calibrate incremental balloon sizes, which correspond to incremental valve sizes. For example, an endobronchial valve may be manufactured in various sizes ranging between 5-9 mm. A medical professional can use a calibration disc that has reference apertures indicative of each of the valve sizes. The medical professional can insert the balloon into each aperture and operate a clear syringe to fill the balloon with the fluid and expand a dimension of the balloon until the balloon contacts the respective reference aperture. The medical professional can then record an amount of the fluid required to expand the balloon to each of the reference apertures to generate a reference table.

After inserting the balloon into a lumen to be sized (e.g., an airway leading to a lung volume to be restricted off from airflow), the medical professional can fill the balloon with fluid until the balloon contacts the internal walls of the lumen. At the point or moment when the balloon contacts the lumen, the medical professional can record the volume of liquid dispensed to expand the balloon. The medical professional can then cross-reference the recorded volume of liquid dispensed to expand the balloon against the reference table to extrapolate (e.g., via mathematical interpolation) the appropriate size for the valve that should be used within the lumen of the patient.

Although commonly used, the sizing procedure discussed above can be tedious and time-consuming due to the multiple calibration steps performed prior to obtaining a lumen size measurement. Thus, the inventor of the present disclosure discovered a need for an accurate, fast, and safe device to measure a dimension of a lumen, airway, or other passageways of a patient. Therefore, the inventor of the present disclosure developed a sizing device that can eliminate the calibration steps with the current valve sizing kit and eliminate several components. In the proposed sizing device, there is no need to pressurize a balloon with a fluid injection, and there are no electrical or electronic controls. In some embodiments, the disclosed sizing device can use the same catheter assembly that can be used to load the valve into lumens, veins, or airway passages of the patient (e.g., the Spiration® Valve System C26N valve deployment system).

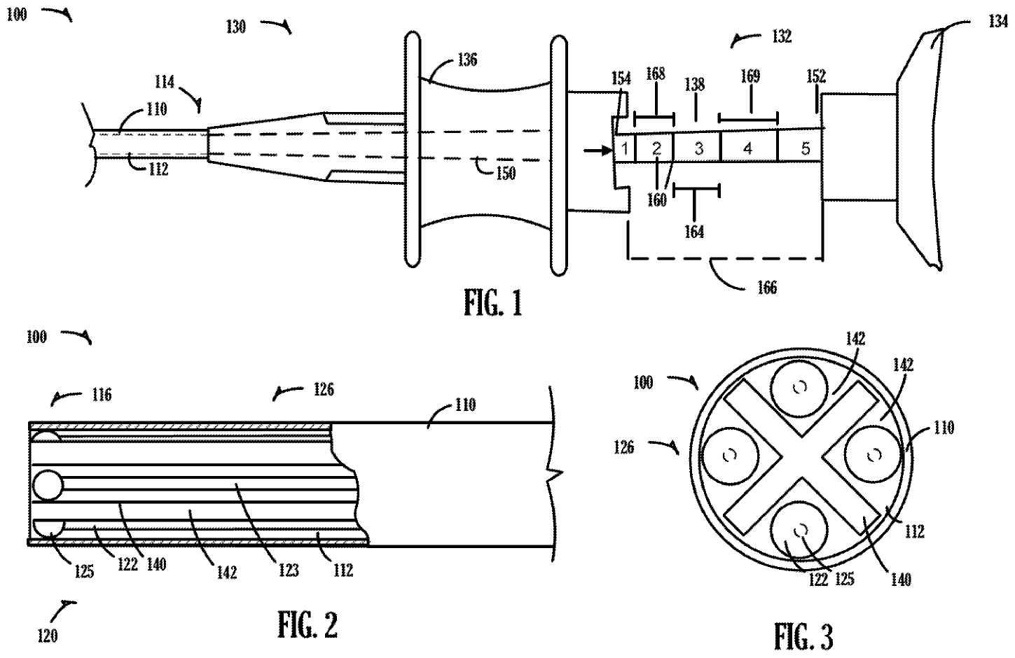

In some embodiments, the sizing device described herein is configured to measure a dimension of an airway (or other anatomical lumen for that matter) in a patient and includes an elongated member, an extendable assembly, and an actuation mechanism. The elongated member can be insertable (e.g., via a working channel of a bronchoscope) into the patient and define a lumen extending from a proximal end to a distal end. The extendable assembly can be insertable into the lumen of the elongated member. The extendable assembly can include elements having a shape-set memory bias. The shape-set memory bias causes the elements to extend radially outward from a central axis (e.g., longitudinal axis) of the elongated member in a controlled manner as the elements of the extendable assembly are extended distally from the distal end of the elongated member. The actuation mechanism can be operably coupled to the extendable assembly and can be configured to move the elements between a retracted position and a plurality of successive deployed positions. In the retracted position, the elements can be disposed within the lumen of the elongated member. In the plurality of deployed positions, the elements can extend distally beyond the distal end of the elongated member and the shape-set memory bias can cause the elements to extend radially outward to define a plurality of testing dimensions. The sizing device will be discussed in more detail herein with reference to FIGS. 1-9.

FIG. 1 illustrates a portion of a sizing device 100, according to an example of the present disclosure. FIG. 2 illustrates a partial cut-away view of a distal portion of an elongated member 110 of the sizing device 100, according to an example of the present disclosure. FIG. 3 illustrates a front view of a distal tip of the elongated member 110 of the sizing device 100, according to an example of the present disclosure. FIGS. 1-3 will be discussed together below to help illustrate an example of the sizing device 100.

The sizing device 100 can be configured to size a lumen (e.g., a vein, artery, airway, or the like) within a patient. In examples, the sizing device 100 can include an elongated member 110, an extendable assembly 120 (first shown in FIG. 2), an actuation mechanism 130, and a core 140 (first shown in FIG. 2, and more clearly shown in FIG. 3).

The elongated member 110 is insertable into a patient. For example, the elongated member 110 can be insertable into a lung of the patient. In examples, the elongated member 110 can be inserted into a patient via a bronchoscope. The elongated member 110 can extend from a proximal end 114 (FIG. 1) to a distal end 116 (FIG. 2). The elongated member 110 can define a lumen 112 (first shown in FIG. 2). The lumen 112 can extend through the elongated member 110 from the proximal end 114 to the distal end 116.

The extendable assembly 120 can be inserted into the lumen 112 of the elongated member 110. The extendable assembly 120 can include elements 122, which can be contained within the lumen 112 by the elongated member 110.

FIGS. 2 and 3 depict a starting point of the sizing device 100, for example, before deployment of the elements 122, where the elements 122 are retracted inside the lumen 112 of the elongated member 110. As described in more detail below, as the extendable assembly 120 extends such that the elements 122 extend beyond the distal end 116 of the elongated member 110, the shape-set memory bias causes the elements to extend radially from the longitudinal axis—where the amount of radial extension of the elements is a pre-calibrated function of the amount distal extension of the proximal handle 134 or other manipulation of the actuation mechanism 130. The elements 122 can include an elongated portion 123 and an end portion 125. The elongated portion 123 can be cylindrical, a rectangular prism, or any other shape that can be elongated and attached to the end portion 125. The end portion 125 can be round or can form an end of the elements 122 such that the end portion 125 can contact an inside of a lumen or airway of the patient. In an example, the elongated portion 123 can include an austenite finish (of a Nitinol material) such that the elements 122 can extend radially outward on one or more planes. In another example, the entirety of the elements 122 can include an austenite finish. The extendable assembly 120 and the elements 122 will be discussed in more detail herein.

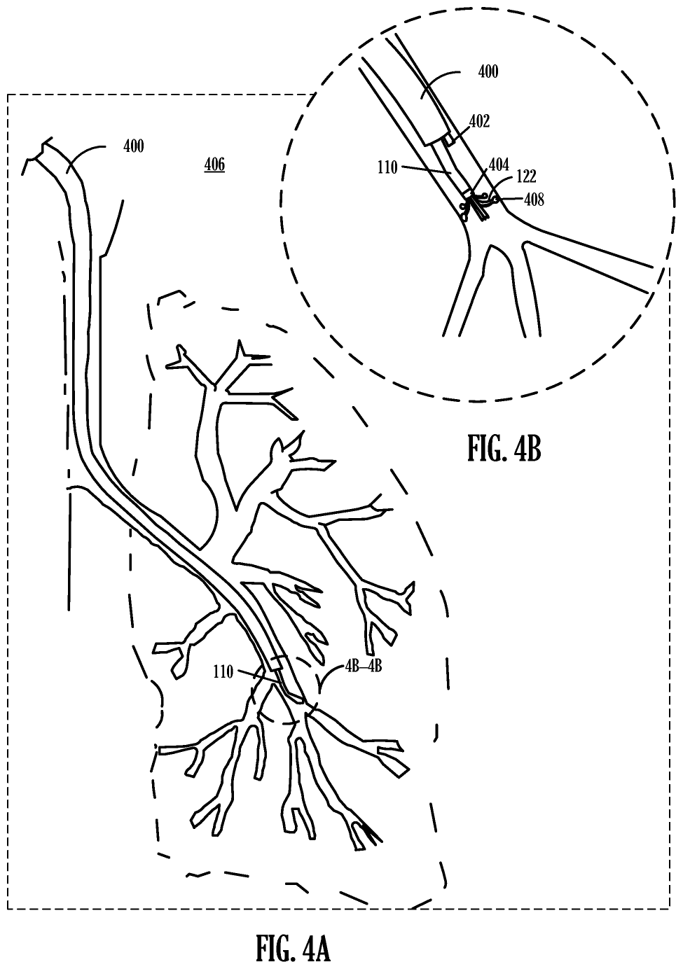

The actuation mechanism 130 can be operably coupled to the extendable assembly 120 and can be configured to move the elements 122 between a retracted position 126 and a plurality of deployed positions 128. The retracted position 126 is shown in FIGS. 2, 3, and 8. The deployed positions 128 are shown in FIGS. 4B, 5A, 5B, 6A, and 6B. In the retracted position 126 the elements 122 are held within the lumen 112 by the elongated member 110. The actuation mechanism 130 can include a handle portion 132, which can be attached to the proximal end 114 of the elongated member 110. The handle portion 132 can include a proximal handle 134, a slider handle 136, and a rod 138.

The proximal handle 134 can define a proximal end of the sizing device 100. The slider handle 136 can also include a working lumen 150 (shown in phantom in FIG. 1) extending therethrough. The working lumen 150 can be connected to the lumen 112 of the elongated member 110. The rod 138 can extend through the working lumen 150 and within the lumen 112. The rod 138 can include a first end section 152 and a second end section 154. The first end section 152 can be coupled to the proximal handle 134, and the second end section 154 can be coupled to the second end portion 196 of the base 190 (the second end portion 196 and the base 190 are shown in FIG. 7). Therefore, moving the proximal handle 134 toward the slider handle 136 can translate the rod 138 with the working lumen 150 and the lumen 112 to extend the elements 122 past the distal end 116 of the elongated member 110, and moving the proximal handle 134 away from the slider handle 136 can translate the rod 138 to retract the elements 122 within the elongated member 110.

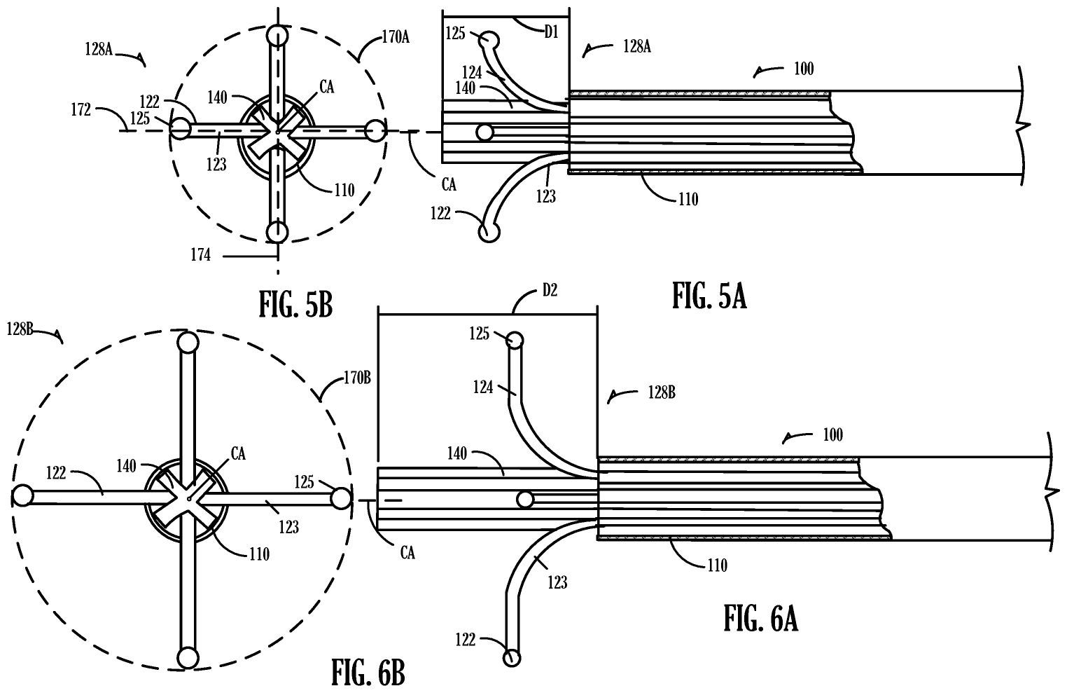

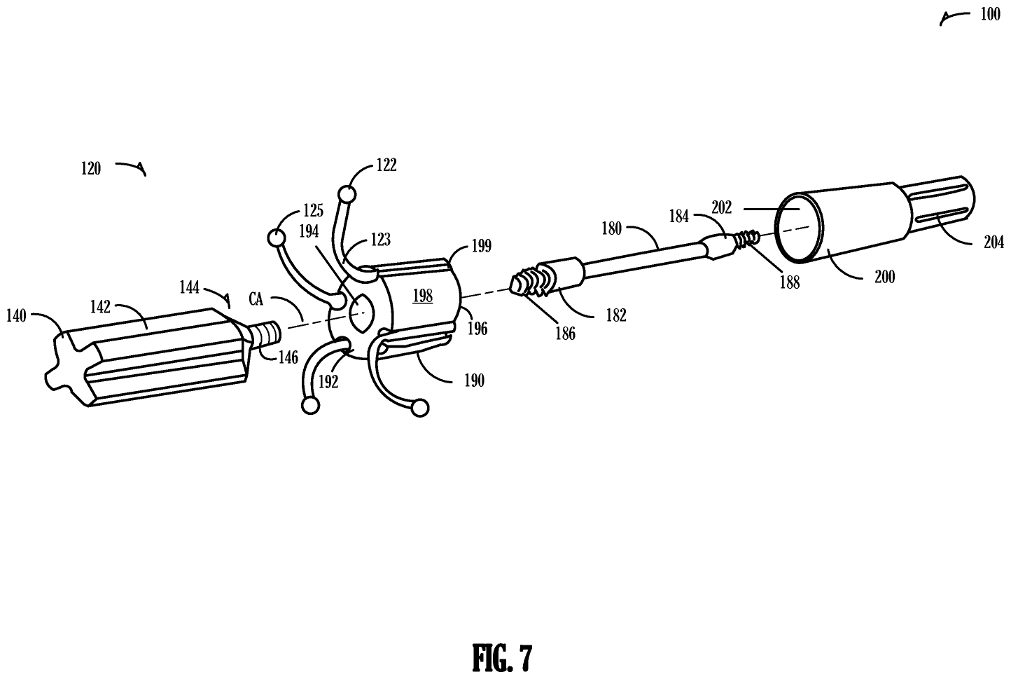

As shown in FIG. 1, the rod 138 can include a plurality of visual indicia 160. As shown in FIG. 1, the visual indicia 160 can be a symbol, for example, a numeric character. In examples, the visual indicia 160 can be alphanumeric characters, symbols, signs, or any other indicia that can be used to distinguish the different indicia as they correspond to the testing dimensions 170. In examples, each visual indicium of the plurality of visual indicia 160 can correspond to one of a plurality of testing dimensions 170. A spacing 164 between adjacent visual indicia of the plurality of visual indicia 160 can correspond to a stroke length 166 required to radially extend the elements 122 from one of the plurality of testing dimensions 170A (shown in FIG. 5B) to another one of the plurality of testing dimensions 170B (shown in FIG. 6B). Stated alternatively, advancing the rod 138 by successive increments corresponding to the visual indicia 160 causes the elements 122 to advance radially by successive increments corresponding to the testing dimensions 170.

In some examples, the stroke length 166 can be a distance the proximal handle 134 moves relative to the slider handle 136 when moving between adjacent visual indicia 160. In some examples, a first spacing 168 between adjacent visual indicia on the second end section 154 of the rod 138 can be smaller than a second spacing 169 between adjacent visual indicia on the first end section 152 of the rod 138 to increase the stroke length 166 required as the plurality of testing dimensions 170 increase. In another example, the first spacing 168 between adjacent visual indicia on the second end section 154 of the rod 138 can be larger than a second spacing 169 between adjacent visual indicia on the first end section 152 of the rod 138 to decrease the stroke length 166 required as the plurality of testing dimensions 170 increase.

In an example, the spacing 164 between adjacent visual indicia of the plurality of visual indicia 160 can vary along the rod 138. As shown in FIG. 1, the spacing 164 between adjacent visual indicia of the plurality of visual indicia 160 can increase as the visual indicia 160 increases. Thus, the spacing 164 can increase as the rod 138 extends toward the proximal handle 134. The spacing 164 increasing as the rod 138 extends toward the proximal handle 134 increases an amount of translation (e.g., the stroke length 166) required of the proximal handle 134 relative to the slider handle 136 as the testing dimensions 170 increases. The increase amount of stroke length 166 between the proximal handle 134 and the slider handle 136 as the testing dimensions 170 increases can help the medical professional slowly increase the testing dimensions 170.

In another example, the spacing 164 can decrease as the rod 138 extends toward the proximal handle 134. The spacing 164 decreasing as the rod 138 extends toward the proximal handle 134 increases an amount of translation (e.g., the stroke length 166) required to enter the first testing dimensions 170, which can provide the medical professional a buffer between when the sizing device 100 first extends outside of the elongated member 110 and when the elements 122 begin forming the testing dimensions 170. The testing dimensions 170 can vary, for example, the testing dimensions 170 can be between 4 and 9 mm. In another example, the testing dimensions 170 can be between 0.5 and 11 mm. In yet another example, the sizing device 100 can be altered to accommodate the testing dimensions 170 required to measure the lumen or airway of the patient.

The core 140 can be insertable into the lumen 112 and can be configured to guide the elements 122 through the lumen 112. The core 140 can include tracks 142, and each track of the tracks 142 can be configured to guide one of the elements 122 through the lumen 112 and prevent contact with other elements 122. As shown at least partially in each of FIGS. 2, 3, 5A, 5B, 6A, 6B, and 7, the core 140 can be an extruded cross shape. The extruded rigid cross-carrier of the core 140 allows the core 140 to capture and position each of the elements 122 into one of the tracks 142. Thus, the core 140 can self-center within the lumen 112 because each one of the tracks 142 can guide one of the elements 122 through the lumen 112. The tracks 142 also prevent the elements 122 from engaging or crisscrossing each other, which helps ensure predictability of the use of the sizing device 100 without the elements 122 jamming up within the lumen 112.

FIG. 4A illustrates a schematic diagram of an example sizing device within a lung of a patient, according to an example of the present disclosure. FIG. 4B illustrates an enlarged view of a portion of the sizing device of FIG. 4A indicated by label 4B-4B, according to an example of the present disclosure. FIGS. 4A and 4B will be discussed together below.

As shown in FIG. 4A, the sizing device 100 can be inserted into the lungs of a patient via a bronchoscope 400. In examples, the bronchoscope can also include a camera 402 that extends through the bronchoscope 400 and can see the sizing device 100 as the sizing device 100 extends from the bronchoscope 400. The elongated member 110 of the sizing device 100 can extend from a distal end of the bronchoscope 400 to further extend into the airways of a patient. As shown in FIG. 4B, the sizing device 100 can be activated such that the elements 122 extend beyond the distal end 116 of the elongated member 110 until the elements 122 contact an inside of the lumen or airway.

As shown in FIG. 4B, the distal end 116 of the elongated member 110 can include an electromagnetic sensor or a magnetic coil 404, which can detect a position of the magnetic coil 404 relative to an electromagnetic field 406 to determine a position of the elongated member 110 within the body. In examples, the magnetic coil 404 can include two wires that extend therefrom such that the electromagnetic field 406 can determine the location of the magnetic coil 404 (and the distal tip of the elongated member 110) within the electromagnetic field 406. The two wires of the magnetic coil 404 allow the magnetic coil 404 to sense the electromagnetic field 406 such that the magnetic coil 404 can determine the six degrees of freedom as the sizing device 100 moves within the patient.

As also indicated in FIG. 4B, each of the elements 122 can include a capacitance sensor 408. For example, a distal end of the elements 122 can include a capacitive coating that can communicate with a sensor, controller, or processor that can receive the signal from the elements 122. The capacitive coating on the distal portion of the elements 122 can include two conductive layers with a dielectric layer therebetween. The capacitance sensor 408 can help determine when each of the elements 122 contacts the inside of a lumen or airway of a patient. For example, the capacitance sensor 408 can be configured to transmit a signal when each of the elements 122 are in contact with an inside of the lumen or airway of the patient. In examples, the capacitance sensor 408 can send a separate signal which can indicate when each of the elements 122 contacts the inside of the lumen or airway. The capacitance sensor 408 can be connected to a controller that can generate an alert or a signal to notify the medical professional when the elements 122 have contacted the inside of the lumen or airway.

FIG. 5A illustrates a side view of a partial cut-away view of a distal end 116 of the elongated member 110 of the sizing device 100 in a first position 128A of the plurality of deployed positions 128, according to an example of the present disclosure. FIG. 5B illustrates a front view of a distal end of the elongated member 110 of the sizing device 100 in the first position 128A of the plurality of deployed positions 128, according to an example of the present disclosure. FIGS. 5A and 5B will be discussed together below.

As shown in FIGS. 2 and 3, and discussed herein, the elements 122 can be disposed within the lumen 112 when the sizing device 100 is in the retracted position 126. In contrast, as shown in FIGS. 5A and 5B, the elements 122 can extend beyond the distal end 116 of the elongated member 110 in the deployed positions 128. For example, as shown in FIGS. 5A and 5B, the elements 122 can extend, and the end portion 125 can define a first testing diameter 170A as the core 140 is extended a first distance D1 from the distal end 116 of the elongated member 110.

In some examples, the elements 122 can also include a shape-set memory bias 124. The shape-set memory bias 124 can be configured to extend the elements 122 radially outward from a central axis CA of the elongated member 110. For example, the elements 122 can include an austenite finish that can configure the shape-set memory bias 124 of the elements 122 such that the elements 122 can expand radially outward from the central axis CA at varying rates as the elements 122 extend out the distal end 116 of the elongated member 110. As shown in FIG. 5B, some of the elements 122 can extend from the elongated member 110 along a first plane 172, and others of the elements 122 can extend along a second plane 174. In examples, the first plane 172 can intersect with the second plane 174 around the central axis CA of the sizing device 100.

FIG. 6A illustrates a side view of a partial cut-away view of a distal end 116 of the elongated member 110 of the sizing device 100 in a second position 128B of a plurality of deployed positions 128, according to an example of the present disclosure. FIG. 6B illustrates a front view of a distal end 116 of the elongated member 110 of the sizing device 100 in the second position 128B of the plurality of deployed positions 128, according to an example of the present disclosure. FIGS. 6A and 6B will be discussed together below.

As shown in FIGS. 6A and 6B, the elements 122 can extend, and the end portion 125 can define a second testing diameter 170B as the core 140 is extended a second distance D2 from the distal end 116 of the elongated member 110. As discussed herein, the elements 122 can also include a shape-set memory bias 124. The shape-set memory bias 124 can be configured to extend the elements 122 radially outward from a central axis CA of the elongated member 110. For example, the elements 122 can include an austenite finish that can configure the shape-set memory bias 124 of the elements 122 such that the elements 122 can expand radially outward from the central axis CA at varying rates as the elements 122 extend out the distal end 116 of the elongated member 110. As shown in FIG. 6B, some of the elements 122 can extend from the elongated member 110 along a first plane 172, and others of the elements 122 can extend along a second plane 174. In examples, the first plane 172 can intersect with the second plane 174 around the central axis CA of the sizing device 100.

The austenite finish of the elements 122 can be altered to change the shape-set memory bias 124 of the elements 122. For example, the austenite finish of the elements 122 can be altered to have a greater rate of dimensional increase as the elements 122 first extend from the distal end 116 of the elongated member 110 and a lower rate of dimensional increase as the elements 122 extend from the distal end 116 of the elongated member 110 toward the max limit of the testing dimensions 170. In yet another example, the austenite finish can be such that the shape-set memory bias 124 has a very rapid radial growth rate when the elements 122 first leave the distal end 116 of the elongated member 110, then a lower rate of radial growth rate in a middle portion of the operation cycle, and a higher rate of radial growth rate toward the end of the operation cycle. Because the austenite finish can be adjusted, the shape-set memory bias 124 of the elements 122 can be customized to match the preferences of the medical professional.

FIG. 7 illustrates an exploded view of a portion of the sizing device 100, according to an example of the present disclosure. An example of how the sizing device 100 can be assembled is shown in FIG. 7. As shown in FIG. 7, the sizing device 100 can include a flex cable 180 and a housing 200. The flex cable 180 can be configured to operably connect the extendable assembly 120 to the actuation mechanism 130.

The extendable assembly 120 can include a base 190. The base 190 can include a first end portion 192 and a second end portion 196. The base 190 can also include a body 198, the body 198 extending between the first end portion 192 and the second end portion 196. A proximal portion 199 of each element of the elements 122 can be attached to the body 198 of the base 190. For example, the proximal portion 199 of each element of the elements 122 can be welded to the body 198. In another example, each element of the elements 122 can be fused, glued, adhered, fixated, or attached to the body 198. In one example, the elements 122 can be attached to the body 198 near the first end portion 192 of the base 190. In another example, the elements 122 can be attached to the body 198 near the second end portion 196 of the base 190.

In some examples, the core 140 can be removably attached to the first end portion 192 of the base 190. As shown in FIG. 7, a proximal end 144 of the core 140 can include an attachment interface 146. The attachment interface 146 can be complementary to an attachment interface 194 of the base 190. In examples, the attachment interface 146 and the attachment interface 194 can be attached to each other via a press fit assembly, crimped, glued, any combination thereof, or the like.

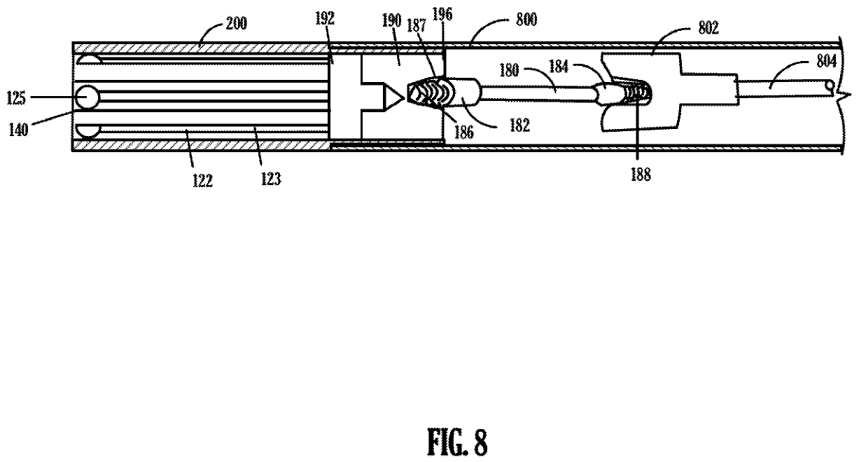

A first end 182 of the flex cable 180 can be removably attached to the second end portion 196 of the base 190. For example, the first end 182 can include threads 186 that can be complementary to threads 187 (shown in FIG. 8) formed therein the second end portion 196 of the base 190. The first end 182 can also be removably to attached to the base 190 via a pin, or any other removable fixture that can removably secure the flex cable 180 to the base 190.

A second end 184 of the flex cable 180 can be removably attached to the second end section 154 of the rod 138. For example, the second end 184 can include threads 186 that are complementary to threads formed therein the second end section 154 of the rod 138. The second end 184 can also include threads 188 that can be complementary to threads formed in the second end section 154 of the rod 138. The second end 184 can also be removably attached to the second end section 154 of the rod 138 via a pin, or any other removable fixture that can removably secure the flex cable 180 to the rod 138.

The housing 200 can be configured to surround the extendable assembly 120. The housing 200 can include a lumen 202 that extends therethrough and can be connected to the lumen 112 of the elongated member 110. The housing 200 can include crushed ribs 204 that can be configured to fit within the distal end 116 of the elongated member 110. For example, the crushed ribs 204 can be configured to compress as the housing 200 is inserted into the distal end 116 of the elongated member 110 to removably attach the housing 200 to the elongated member 110. As the crushed ribs 204 compress, the crushed ribs 204 can generate a radially outward force that can increase a frictional force between the housing 200 and the elongated member 110 to hold the housing 200 within the elongated member 110. As such, the extendable assembly 120 can translate within the housing 200 to extend the elements 122 outside the housing 200 and radially outward from the central axis CA.

To assemble the portion of the sizing device 100 shown in FIG. 7, the attachment interface 146 on the proximal end 144 of the core 140 can be attached (e.g., press fit, glued, or otherwise attached) into the attachment interface 194 on the first end portion 192 of the base 190 to attach the core 140 to the base 190. The threads 186 on the first end 182 of the flex cable 180 can be screwed into the threads formed in the second end portion 196 of the base 190 to attach the flex cable 180 to the base 190. Then the flex cable 180, the base 190, and the core 140 can be slid into the housing 200 until the core 140 and the elements 122 are completely within the housing 200. The threads 188 of the second end 184 can be attached to the rod 138, and the housing 200 can be slid into the distal end 116 of the elongated member 110.

FIG. 8 illustrates a partial cross-sectional view of a sizing device 100, according to an example of the present disclosure. The sizing device 100 shown in FIG. 8 can be used with an existing catheter, for example, the actuation mechanism 130 of the sizing device 100 can be an existing catheter. Here, the sizing device 100 can be used with the C26N catheter used to insert the valve into an airway of a patient. In another example, the sizing device 100 shown in FIG. 8, can be modified to work with any other catheter used to insert a medical device within a patient. When the sizing device 100 is used with a catheter, the catheter can include a shaft 800, a modified tip stabilizer 802, and an existing wire 804 of the catheter.

The housing 200 can be inserted into the shaft 800 and the flex cable 180 can be attached to the modified tip stabilizer 802 to attach the sizing device 100 to the catheter. The catheter can then be used to actuate the sizing device 100 to extend the elements 122 beyond the housing 200 and radially outward to define a testing diameter.

As discussed herein, the sizing device 100 includes four elements of the elements 122. In examples, the sizing device 100 can include any number of the elements 122. For example, the sizing device 100 can include 1-10 of the elements 122. In another example, the sizing device 100 can include 10 or more of the elements 122. As discussed herein, the core 140 can be adjusted to alter a number of the tracks 142 such that there can be one of the tracks 142 for each of the elements 122.

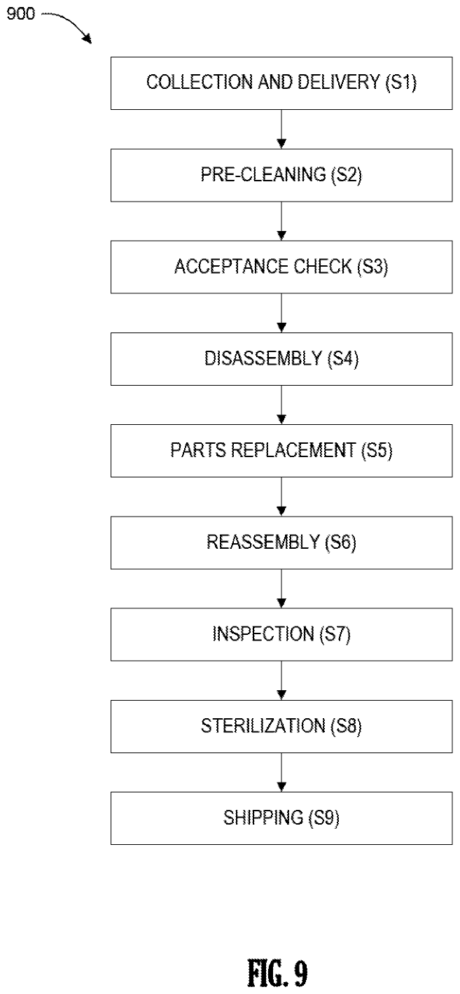

Next, a reprocessing method 900 for the above-described sizing device 100 will be described. FIG. 9 is a flowchart indicating the reprocessing method 900 for the sizing device 100. The sizing device 100 described above may be disposed of after one use or may be repeatedly used a plurality of times. In the case of a configuration that can be repeatedly used a plurality of times, for example, reprocessing method 900 shown in FIG. 9 can be used to reprocess the sizing device 100. An operator who remanufactures collects the used sizing device 100 after it has been used for treatment and transports it to a factory or the like (Step S1). At this time, the used sizing device 100 can be transported in a dedicated container to prevent contamination from the sizing device 100.

Then, the operator can clean and sterilize the collected and transported used sizing device 100 (Step S2). Specifically, in cleaning the sizing device 100, deposits adhering to the distal end 116 of the elongated member 110, the elements 122, the extendable assembly 120, or any other portion of the sizing device 100 can be removed by using a brush or the like. After that, to remove pathogenic microorganisms and the like derived from blood, body fluid, etc., the the distal end 116 of the elongated member 110, the elements 122, the extendable assembly 120, or any other portion of the sizing device 100 can be used with any cleaning solution of isopropanol-containing cleaning agent, proteolytic enzyme detergent, and alcohol. And the the distal end 116 of the elongated member 110, the elements 122, the extendable assembly 120, or any other portion of the sizing device 100 can be cleaned. The cleaning liquid is not limited to the cleaning liquid described above, and other cleaning liquids may be used. Further, in the sterilization of the sizing device 100, to sterilize the pathogenic microorganisms and the like adhering to the the distal end 116 of the elongated member 110, the elements 122, the extendable assembly 120, or any other portion of the sizing device 100, any of high-pressure steam sterilization, ethylene oxide gas sterilization, gamma ray sterilization, hydrogen peroxide and hydrogen peroxide low temperature sterilization can be used. The distal end 116 of the elongated member 110, the elements 122, the extendable assembly 120, or any other portion of the sizing device 100 can be disassemble as discussed in FIGS. 7 and 8. Therefore, the distal end 116 of the elongated member 110, the elements 122, the extendable assembly 120, or any other portion of the sizing device 100 can be easy to clean.

And the operator performs an acceptance check of the used sizing device 100 (Step S3). In detail, the operator checks whether the used sizing device 100 has significant defects or the used sizing device 100 exceed a maximum number of reprocessing.

Next, the operator disassembles the used sizing device 100 (Step S4). Specifically, the operator disassembles the sizing device 100. As discussed in FIG. 7, the core 140 can be removably attached to the base 190, and the base 190 can also be removably attached to the actuation mechanism 130 via the flex cable 180 or the rod 138. Therefore, the sizing device 100 can be easy to disassemble.

After the step S4, some parts are replaced (Step S5). For example, the base 190 and the elements 122 can be replaced if the capacitance coating on the elements 122 can be worn, or if the elements 122 has gone through too many cycles such that the elements 122 lose the shape-set memory bias 124. As discussed above, the base 190 can be removably attached to other components of the sizing device 100. Therefore, there can be advantage that it is easy to replace base 190 and the elements 122 in the Step S5.

After step S5, the operator can assemble a new sizing device 100 (Step S6). In detail, the new sizing device 100 can be assembled as discussed with reference to FIG. 7. As discussed herein, the components of the sizing device 100 are removably attached to each other. Therefore, there is advantage that it is easy to assemble in the Step S6.

In some examples, Step S6 can include adding an identifier to indicate the device has been modified from its original condition, such as a adding a label or other marking to designate the device as reprocessed, refurbished or remanufactured.

After step S6, the operator inspects and tests the newly formed sizing device 100 (step S7). Specifically, the operator who remanufactures can verify that the newly formed sizing device 100 has the same effectiveness and safety as the original product by various functional tests. There is advantage that it is easy to verify in the Step S7.

After Step S7, the operator can sequentially perform sterilization and storage (Step S8), and shipping (Step S9) of the new sizing device 100. In Step S8, in step S7, a sterilization treatment can use a sterilizing gas such as ethylene oxide gas or propylene oxide gas is applied to the new sizing device 100 and the device can be stored in a storage container until use.

Steps S1 to S9 described above are executed to achieve reprocessing of the sizing device 100.

The following, non-limiting examples, detail certain aspects of the present subject matter to solve the challenges and provide the benefits discussed herein, among others.

Example 1 is a sizing device configured to measure a dimension of an airway in a patient, the sizing device comprising: an elongated member insertable into a lung of the patient and defining a lumen extending therethrough from a proximal end to a distal end; an extendable assembly insertable into the lumen, the extendable assembly including wires having a shape-set memory bias, the shape-set memory bias configured to allow the wires to extend radially outward from a central axis of the elongated member; and an actuation mechanism operably coupled to the extendable assembly and configured to move the wires between a retracted position, wherein the wires are disposed within the lumen, and a plurality of deployed positions, wherein the wires extend distally beyond the distal end of the elongated member, wherein in the deployed positions, the shape-set memory bias causes the wires to extend radially outward to define a plurality of testing dimensions.

In Example 2, the subject matter of Example 1 includes, a core insertable into the lumen and configured to guide the wires through the lumen.

In Example 3, the subject matter of Example 2 includes, wherein the core comprises tracks, each track configured to guide one of the wires through the lumen and prevent contact with other wires.

In Example 4, the subject matter of Examples 2-3 includes, wherein the extendable assembly comprises: a base including a first end portion, a second end portion, and a body, the body extending between the first end portion and the second end portion, wherein a proximal portion of each of the wires is attached to the body of the base.

In Example 5, the subject matter of Example 4 includes, wherein the core is removably attached to the first end portion of the base.

In Example 6, the subject matter of Examples 4-5 includes, wherein the actuation mechanism is removably coupled to the second end portion of the base.

In Example 7, the subject matter of Examples 4-6 includes, wherein the actuation mechanism comprises: a handle portion attached to the proximal end of the elongated member, the handle portion including: a proximal handle; a slider handle including a working lumen extending therethrough, the working lumen connected to the lumen of the elongated member; and a rod having a first end section and a second end section, the first end section of the rod coupled to the proximal handle, the second end section of the rod coupled to the second end portion of the base.

In Example 8, the subject matter of Example 7 includes, wherein moving the proximal handle toward the slider handle translates the rod to extend the wires past the distal end of the elongated member, and wherein moving the proximal handle away from the slider handle translates the rod to retract the wires within the elongated member.

In Example 9, the subject matter of Example 8 includes, wherein the rod comprises: a plurality of visual indicia, each visual indicium of the plurality of visual indicia corresponding to one of the plurality of testing dimensions.

In Example 10, the subject matter of Example 9 includes, wherein a spacing between adjacent visual indicia of the plurality of visual indicia varies along the rod.

In Example 11, the subject matter of Example 10 includes, wherein the spacing between adjacent visual indicia of the plurality of visual indicia corresponds to a stroke length required to radially extend the plurality of wires from one of the plurality of testing dimensions to another one of the plurality of testing dimensions.

In Example 12, the subject matter of Example 11 includes, wherein the stroke length is a distance the proximal handle moves relative to the slider handle when moving between adjacent visual indicia.

In Example 13, the subject matter of Example 12 includes, wherein a first spacing between adjacent visual indicia on the second end section of the rod is smaller than a second spacing between adjacent visual indicia on the first end section of the rod to increase the stroke length required as the plurality of testing dimensions increases.

In Example 14, the subject matter of Examples 1-13 includes, a capacitance sensor in communication with distal ends of the wires, the capacitance sensor configured to transmit a signal when each of the wires is in contact with the airway of the patient.

In Example 15, the subject matter of Examples 1-14 includes, wherein the actuation mechanism is a catheter.

Example 16 is a sizing device configured to measure a dimension of a lumen in a patient, the sizing device comprising: an elongated member insertable into the patient and defining a lumen extending therethrough from a proximal end to a distal end; an extendable assembly insertable into the lumen, the extendable assembly including elements having a shape-set memory bias, the shape-set memory bias configured to allow the elements to extend radially outward from a central axis of the elongated member; and an actuation mechanism operably coupled to the extendable assembly and configured to move the elements between a retracted position, wherein the elements are disposed within the lumen, and a plurality of deployed positions, wherein the elements extend distally beyond the distal end of the elongated member, wherein in the deployed positions, the shape-set memory bias causes the elements to extend radially outward to define a plurality of testing dimensions.

In Example 17, the subject matter of Example 16 includes, wherein the extendable assembly comprises: a base including a first end portion, a second end portion, and a body, the body extending between the first end portion and the second end portion, wherein a proximal portion of each of the elements is attached to the body of the base; wherein the actuation mechanism is removably coupled to the second end portion of the base.

In Example 18, the subject matter of Example 17 includes, wherein the actuation mechanism comprises: a handle portion attached to the proximal end of the elongated member, the handle portion including: a proximal handle; a slider handle including a working lumen extending therethrough, the working lumen connected to the lumen of the elongated member; and a rod having a first end section and a second end section, the first end section of the rod coupled to the proximal handle, the second end section of the rod coupled to the second end portion of the base.

In Example 19, the subject matter of Example 18 includes, wherein moving the proximal handle toward the slider handle translates the rod to extend the elements past the distal end of the elongated member, and wherein moving the proximal handle away from the slider handle translates the rod to retract the elements within the elongated member.

Example 20 is a method for reprocessing the sizing device of Example 16, the method comprising: obtaining the sizing device of claim 16; sterilizing the sizing device; and storing the sizing device in a sterile container.

Example 21 is at least one machine-readable medium including instructions that, when executed by processing circuitry, cause the processing circuitry to perform operations to implement of any of Examples 1-20.

Example 22 is an apparatus comprising means to implement of any of Examples 1-20.

Example 23 is a system to implement of any of Examples 1-20.

Example 24 is a method to implement of any of Examples 1-20.

The above-detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

The devices disclosed herein can be designed to be disposed of after a single use, or they can be designed to be used multiple times. In either case, however, the device can be reconditioned for reuse after at least one use. Reconditioning can include a combination of the steps of disassembly of the device, followed by cleaning or replacement of particular pieces, and subsequent reassembly. In particular, the device can be disassembled, and any number of particular pieces or parts of the device can be selectively replaced or removed in any combination. Upon cleaning or replacement of particular parts, the device can be reassembled for subsequent use either at a reconditioning facility, or by a surgical team immediately prior to a surgical procedure. Those of ordinary skill in the art will appreciate that the reconditioning of a device can utilize a variety of different techniques for disassembly, cleaning/replacement, and reassembly. Use of such techniques, and the resulting reconditioned device, are all within the scope of the present application.

Preferably, the invention described herein will be processed before surgery. First a new or used instrument is obtained and, if necessary, cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or higher energy electrons. The radiation kills bacteria on the instrument and in the container. The sterilized instrument can then be stored in the sterile container. The sealed container keeps the instrument sterile until it is opened in the medical facility. The device may also be sterilized using any other technique known in the art, including but limited to beta or gamma radiation, ethylene oxide, or steam.

1. A sizing device configured to measure a dimension of an airway in a patient, the sizing device comprising:
an elongated member insertable into the airway of the patient and defining a lumen extending therethrough from a proximal end to a distal end;
an extendable assembly insertable into the lumen, the extendable assembly including wires having a shape-set memory bias, the shape-set memory bias configured to cause the wires to extend radially outward from a central axis of the elongated member; and
an actuation mechanism operably coupled to the extendable assembly and configured to move the wires between a retracted position, wherein the wires are disposed within the lumen, and a plurality of deployed positions, wherein the wires extend distally beyond the distal end of the elongated member, wherein in the deployed positions, the shape-set memory bias causes the wires to extend radially outward to define a plurality of testing dimensions.

2. The sizing device of claim 1, further comprising:
a core insertable into the lumen and configured to guide the wires through the lumen.

3. The sizing device of claim 2, wherein the core comprises tracks, each track configured to guide one of the wires through the lumen and prevent contact with other wires.

4. The sizing device of claim 2, wherein the extendable assembly comprises:
a base including a first end portion, a second end portion, and a body, the body extending between the first end portion and the second end portion, wherein a proximal portion of each of the wires is attached to the body of the base.

5. The sizing device of claim 4, wherein the core is removably attached to the first end portion of the base.

6. The sizing device of claim 4, wherein the actuation mechanism is removably coupled to the second end portion of the base.

7. The sizing device of claim 4, wherein the actuation mechanism comprises:
a handle portion attached to the proximal end of the elongated member, the handle portion including:
a proximal handle;
a slider handle including a working lumen extending therethrough, the working lumen connected to the lumen of the elongated member; and
a rod having a first end section and a second end section, the first end section of the rod coupled to the proximal handle, the second end section of the rod coupled to the second end portion of the base.

8. The sizing device of claim 7, wherein moving the proximal handle toward the slider handle translates the rod to extend the wires past the distal end of the elongated member, and wherein moving the proximal handle away from the slider handle translates the rod to retract the wires within the elongated member.

9. The sizing device of claim 8, wherein the rod comprises:
a plurality of visual indicia, each visual indicium of the plurality of visual indicia corresponding to one of the plurality of testing dimensions.

10. The sizing device of claim 9, wherein a spacing between adjacent visual indicia of the plurality of visual indicia varies along the rod.

11. The sizing device of claim 10, wherein the spacing between adjacent visual indicia of the plurality of visual indicia corresponds to a stroke length required to radially extend the plurality of wires from one of the plurality of testing dimensions to another one of the plurality of testing dimensions.

12. The sizing device of claim 11, wherein the stroke length is a distance the proximal handle moves relative to the slider handle when moving between adjacent visual indicia.

13. The sizing device of claim 12, wherein a first spacing between adjacent visual indicia on the second end section of the rod is smaller than a second spacing between adjacent visual indicia on the first end section of the rod to increase the stroke length required as the plurality of testing dimensions increases.

14. The sizing device of claim 1, further comprising:
a capacitance sensor in communication with distal ends of the wires, the capacitance sensor configured to transmit a signal when each of the wires is in contact with the airway of the patient.

15. The sizing device of claim 1, wherein the actuation mechanism is a catheter.

16. A sizing device configured to measure a dimension of a lumen in a patient, the sizing device comprising:
an elongated member insertable into the patient and defining a lumen extending therethrough from a proximal end to a distal end;
an extendable assembly insertable into the lumen, the extendable assembly including elements having a shape-set memory bias, the shape-set memory bias configured to allow the elements to extend radially outward from a central axis of the elongated member; and
an actuation mechanism operably coupled to the extendable assembly and configured to move the elements between a retracted position, wherein the elements are disposed within the lumen, and a plurality of deployed positions, wherein the elements extend distally beyond the distal end of the elongated member, wherein in the deployed positions, the shape-set memory bias causes the elements to extend radially outward to define a plurality of testing dimensions.

17. The sizing device of claim 16, wherein the extendable assembly comprises:
a base including a first end portion, a second end portion, and a body, the body extending between the first end portion and the second end portion, wherein a proximal portion of each of the elements is attached to the body of the base;
wherein the actuation mechanism is removably coupled to the second end portion of the base.

18. The sizing device of claim 17, wherein the actuation mechanism comprises:
a handle portion attached to the proximal end of the elongated member, the handle portion including:
a proximal handle;
a slider handle including a working lumen extending therethrough, the working lumen connected to the lumen of the elongated member; and
a rod having a first end section and a second end section, the first end section of the rod coupled to the proximal handle, the second end section of the rod coupled to the second end portion of the base.

19. The sizing device of claim 18, wherein moving the proximal handle toward the slider handle translates the rod to extend the elements past the distal end of the elongated member, and wherein moving the proximal handle away from the slider handle translates the rod to retract the elements within the elongated member.
